(19) United States
(12) Patent Application Publication
(10) Pub. No.: US 2023/0157585 A1
(43) Pub. Date: May 25, 2023

(54) Spatial Hearing Measurement System

(71) Applicant: Sensimetrics Corporation, Gloucester, MA (US)

(72) Inventors: Thomas E. Von Wiegand, Billerica, MA (US); Patrick M. Zurek, Arlington, MA (US)

(21) Appl. No.: 17/991,229

(22) Filed: Nov. 21, 2022

Related U.S. Application Data
(60) Provisional application No. 63/281,799, filed on Nov. 22, 2021.

(51) Int. Cl. A61B 5/12; G08B 5/36; H04R 5/02; A61B 5/11; A61B 5/00

(52) U.S. Cl. CPC A61B 5/123; G08B 5/36; H04R 5/02; A61B 5/1114; A61B 5/1126; A61B 5/742; A61B 2562/04

(57) ABSTRACT

A measurement system includes a number of audio transducers configured in a first arrangement for acoustic testing of a subject in a testing position, one or more sensors for collecting measurement data characterizing a position of a subject's head relative to the audio transducers, a controller for processing the measurement data to determine feedback data characterizing a difference between the position of the subject's head and the testing position, and a feedback indicator for presenting the feedback data to the subject.

### CROSS REFERENCE TO RELATED APPLICATIONS

This application claims priority to U.S. application Ser. No. 63/281,799, filed on Nov. 22, 2021, the contents of which is hereby incorporated by reference in its entirety.

### BACKGROUND OF THE INVENTION

This invention relates to measurement of a subject's hearing, and more particularly to measurement of spatial (e.g., directional) characteristics of hearing.

Hearing tests are used to evaluate the sensitivity of a subject's sense of hearing. One type of hearing test measures a subject's hearing sensitivity (e.g., an absolute threshold for hearing) at different frequencies. Headphones are often used to administer hearing sensitivity tests.

Another type of hearing test measures spatial hearing. Spatial hearing is an ability of a subject to use spatial (e.g., directional) cues, such as interaural time difference, to determine where a sound originates from in space. A subject's spatial hearing ability may affect their ability to understand speech in the presence of background noise. Spatial hearing tests are sometimes administered by large test systems, for example, by playing stimulus tones from two loudspeakers in different spatial arrangements relative to the subject.

### SUMMARY OF THE INVENTION

Very generally, aspects described herein address the need for a standardized spatial-hearing test system that can be fit easily in limited space, for example, within typical audiometric sound booths. There is demand for such a system in both the military and civilian arenas. Because good spatial hearing is vital for many military (as well as civilian police and emergency) personnel, a reliable system is needed both for testing the spatial hearing abilities of personnel and for assessing the impact of head-worn equipment on those abilities. Parallel applications arise in civilian audiology clinics with the need to assess the spatial dimension of patients' hearing and the effects that hearing aids and cochlear implants have on those abilities.

Current approaches to testing of spatial hearing generally use an array of loudspeakers that are placed at a distance from a subject. Because of the size of such testing systems, they can take up valuable floor space in a clinic. Ideally, the testing system would be housed in a very quiet space, such as an audiometric sound booth. But because generally used clinical booths are relatively small, the array must be very compact to interfere minimally with other activities in the booth. The size of the array (the distance from listener to loudspeakers) is an issue with a compact array because placing sources too close to the listener results in distortions of the interaural (between-ear) acoustic differences that cue sound location.

Aspects described herein relate to a multi-loudspeaker spatial hearing test system that is sized and shaped to fit in a standard sound booth. In some examples, because of the compact nature of the test system, a position of a subject's head relative to the test system must be carefully controlled to ensure accurate test results. To this end, the test system includes a feedback system that senses a subject's head position and provides feedback to the subject (e.g., using a visible indicator) to assist the subject in correctly positioning their head.

In a general aspect, a measurement system includes a number of audio transducers configured in a first arrangement for acoustic testing of a subject in a testing position, one or more sensors for collecting measurement data characterizing a position of a subject's head relative to the audio transducers, a controller for processing the measurement data to determine feedback data characterizing a difference between the position of the subject's head and the testing position, and a feedback indicator for presenting the feedback data to the subject.

Aspects may include one or more of the following features.

The audio transducers may be arrayed on a horizontal plane intersecting with the testing position. The audio transducers may be arranged symmetrically over a range of between ±15 degrees and ±30 degrees relative to a 0-degree angle relative to the testing position. The audio transducers may be spaced apart with a first audio transducer oriented with a 0-degree angle relative to the testing position, second and third audio transducers oriented with a ±15 degree angle relative to the testing position, fourth and fifth audio transducers oriented with a ±30 degree angle relative to the testing position, and sixth and seventh audio transducers oriented with a ±60 degree angle relative to the testing position.

A first sensor of the one or more sensors may be configured to sense the left-hand side of the subject's head, a second sensor of the one or more sensors may be configured to sense the right-hand side of the subject's head, and a third sensor may be configured to sense a front side of the subject's head. The one or more sensors may include one or more optical distance measurement devices. The feedback indicator may be configurable to provide controllable patterns of illumination. The feedback indicator may include an LED array. The feedback data presented to the subject may indicate actions that the subject can take to reduce the difference between their head position and the testing position.

The controller may be configured to conduct a test when the subject's head is in the testing position, the test including causing emission of acoustic energy from at least some of the plurality of transducers. Causing emission of the acoustic energy may include supplying one or more signals to the at least some transducers. The controller may be configured to modify the one or more signals to simulate reverberation in the emitted acoustic energy. The measurement system may be sized and shaped such that the plurality of audio transducers fit in a sound booth. The plurality of audio transducers may include a plurality of loudspeakers.

In another general aspect, a method for testing spatial hearing includes sensing a position of a subject's head relative to the measurement system above, providing feedback to the subject based on the sensing of the position, providing acoustic stimuli from loudspeakers of the measurement system, and receiving responses to the acoustic stimuli from the subject.

Aspects may have one or more of the following advantages.

Among other advantages, aspects allow for spatial hearing measurement tests without requiring the use of headphones, allowing for testing of children, military headsets, helmets, and headgear, and hearing aids.

Previous spatial hearing test systems are generally large systems located in an office setting and outside of a sound booth. Aspects described herein are advantageously compact enough to fit in a sound booth (e.g., the system is 34″ from end to end). Sound booths are advantageously quieter than office settings and include fewer reflections.

Aspects advantageously locate the center of the user's head at least 50 cm from each loudspeaker, which is a distance that can be used without incurring unnatural head diffraction effects from being too close to the source.

Other features and advantages of the invention are apparent from the following description, and from the claims.

### BRIEF DESCRIPTION OF THE DRAWINGS

FIG. 1 is a spatial hearing measurement system.

FIG. 2 is a schematic diagram of the spatial hearing measurement system.

FIGS. 3A-3B show a feedback mechanism of the spatial hearing measurement system.

### DETAILED DESCRIPTION

### 1 Overview

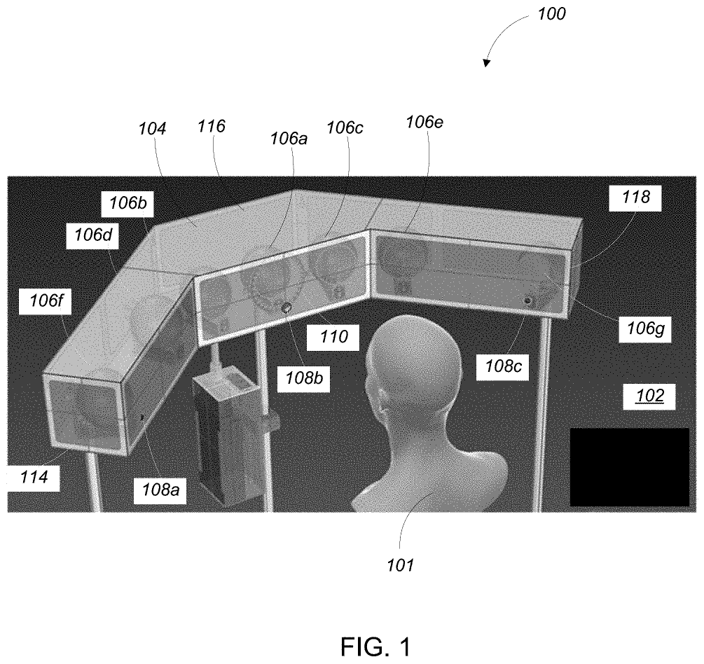

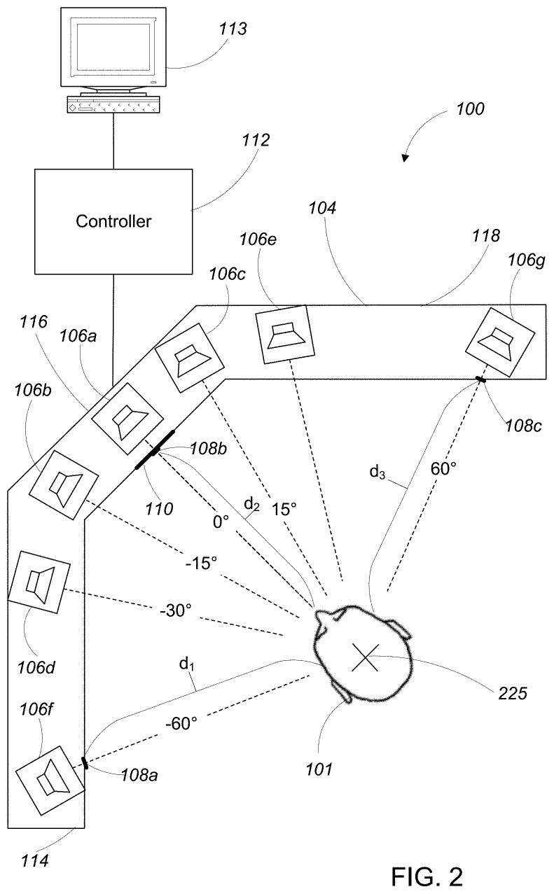

Referring to FIGS. 1 and 2, a spatial hearing test system 100 is configured for administering a spatial hearing test to a subject 101. The test system 100 is sized and shaped to fit in a confined environment 102 (e.g., an audio booth, not shown) and includes a cabinet 104, loudspeakers 106a-g, sensors 108a-c, a feedback indicator 110, a controller 112, and a computer 113 (e.g., a clinician's workstation).

The cabinet 104 is substantially u-shaped and includes a left section 114, a center section 116, and a right section 118. When the subject 101 is positioned in the test system 100, the left section 114 faces the left-hand side of the subject's head, the center section 116 faces the front of the subject's head, and the right section 118 faces the right-hand side of the subject's head.

### 2 Loudspeaker Placement

The testing system is installed such that the loudspeakers 106a-f are arrayed about the subject's head with a first loudspeaker 106a positioned in the center section 116 of the housing 104. When the subject 101 is properly positioned in the test system 100, the first loudspeaker 106a is aligned with the center of the subject's face, referred to as the 0° or “center” alignment. A second loudspeaker 106b is positioned in the center section 116 and oriented at a −15° angle from the center alignment. A third loudspeaker 106c is positioned in the center section 116 and is oriented at a +15° angle from the center alignment.

A fourth loudspeaker 106d is positioned in the left section 114 and is oriented at a −30° angle from the center alignment. A fifth loudspeaker 106e is positioned in the right section 118 and is oriented at a +30° angle from the center alignment. A sixth loudspeaker 106f is positioned in the left section 114 and is oriented at a −60° angle from the center alignment. A seventh loudspeaker 106g is positioned in the right section 118 and is oriented at a +60° angle from the center alignment.

Very generally, the configuration of the loudspeakers described above is confined to a horizontal plane that is substantially aligned with a testing position, which is aligned with the user's ears when the user is properly positioned. This is because users with normal hearing typically have an excellent ability to sense direction in the left-right dimension but a poor ability to sense direction in the vertical dimension.

Another factor that influences the loudspeaker configuration is the approximate front/back symmetry of the human head. Due to that symmetry, acoustic cues for sound localization are approximately equivalent for azimuthal angles of α and 180°−α (where 0° is straight-ahead of the listener). As such, including loudspeakers for testing localization in the rear half-plane would be redundant and would also increase the footprint of the array considerably. So, the loudspeakers in the array are confined to the frontal half of the horizontal plane.

Yet another factor that influences the loudspeaker configuration is that examination of interaural cues has shown that they are maximal (or nearly so) for an angle of ±60°. So, the loudspeakers in the array are confined to that angular range.

The 120° range of loudspeaker placement allows for clinical testing of sound localization ability and speech reception in noise with speech and noise over that range. A minimal source separation of 15° is sufficient for assessing localization ability for real-world function.

### 3 Feedback System

In general, a distance from the user's head to the loudspeakers is small, for example, 50 cm in the embodiment illustrated in FIGS. 1-2. Because of that small distance, relatively small head movements could result in large changes in the sound levels at the user's ears. To maintain the user's head in the testing position 225 without resorting to physical devices (e.g., chin or head rest), the system employs optical distance sensors 108a-c to measure the user's head position. This measurement is then used to give feedback to the user on their head position and how to correct it.

In some examples, there are three sensors 108a-c in the housing 104, with a first sensor 108a in the left section 114, a second sensor 108b in the center section 116, and a third sensor 108c in the right section 118.

Each of the three sensors 108a-c emits an optical signal and measures an amount of time it takes for the optical signal to reflect off the subject's head and back to the sensor. An output of each of the sensors 108a-c is provided to the controller 112, which uses the outputs to determine a first distance, d₁ between the user's head and the first sensor 108a, a second distance, d₂ between the user's head and the second sensor 108b, and a distance, d₃ between the user's head and the third sensor 108c.

The controller 112 uses the distances, d₁, d₂, and d₃ to estimate a position of the subject's head relative to the spatial hearing system 100. The controller 112 then compares the estimated position of the subject's head to the testing position 225 to determine a measure of deviation from the testing position (e.g., 5 cm too close to the array and 3 cm too far to the left).

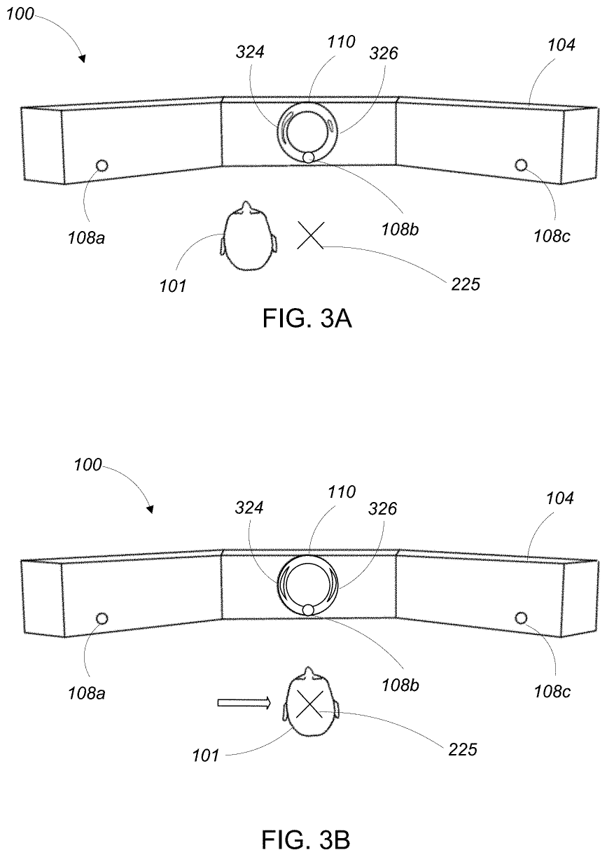

Referring to FIGS. 3A-3B, the determined measure of deviation is used to display feedback to the user 101 via the feedback indicator 110. In some examples, the feedback indicator 110 is a circular LED array. In FIG. 3A, the measure of deviation indicates that the user's head is too far to the left, so the feedback indicator 110 activates more LEDs on its left-hand side 324 than on its right-hand side 326 (indicating to the user that they are too far left).

Referring to FIG. 3B, as the user 101 moves their head from left to right, the controller 112 reduces the number of LEDs active on the left-hand side 324 of the feedback indicator 110 and increases the number of LEDs active on its right-hand side 326 to reflect the user's head movement. When the user's head is in the testing position 225 (as in FIG. 3B), the number of LEDs active on the left-hand side 324 of the indicator 110 is substantially the same as the number of LEDs active on the right-hand side 326 of the indicator 110. More generally, the feedback indicator 110 can be used to guide the user 101 to the testing position 225 in the left-to-right (or right-to-left) direction and in the front-to-back (or back-to-front) direction. Once the user is in the testing position 225, the system can conduct a test.

In some examples, some or all of the loudspeakers described above have associated visual indicators (e.g., LEDs, not shown). The visual indicators can be used to inform a user of locations of one or more loudspeakers (e.g., the loudspeaker(s) being used in a particular hearing test). This is another form of feedback, where the indicators direct a user's attention to one or more specific loudspeakers that indicated by the system 100.

### 4 Applications

In general, for the applications described below, the system 100 causes emission of audio stimuli to test subjects and the test subjects provide responses to the stimuli by, for example, verbally responding to a technician or interacting with a computer system to enter a response.

One application of the system is localization testing. Localization is the ability to determine the direction to a sound source. This ability can be measured very directly and quickly by presenting sound from one of the seven loudspeakers and asking the user/listener to respond by reporting the direction (e.g., a verbal response or a response by interacting with an input device of a computing system). The listener's responses will form a pattern indicating errors and biases in localization. Because normal-hearing listeners' localization ability in the left/right dimension is very acute, with a just-noticeable difference of about 1 degree from straight-ahead, the localization task distinguishing which of the seven loudspeakers of the system 100 is emitting a signal is very easy. The array configuration is sufficient, however, to disclose clinically meaningful degradations in localization ability using combinations of output from the multiple speakers.

Another application is speech in noise testing. Speech-in-noise testing assesses the benefit a listener gains from spatial separation between a target speech source and a masking noise source. This ‘spatial release from masking’ (SRM) is measured by delivering a list of speech test items, such as sentences, from one location and a masking noise from another location. Performance in that condition is compared to that obtained in the condition where the speech and noise arise from the same location.

The system 100 also provides improved sound level stability for threshold testing. Maintaining a calibrated sound level is important when measuring hearing. But keeping a calibrated sound level at the listener's location is problematic, especially at high frequencies where, when testing with a single loudspeaker as is usually done, small head movements can cause large changes in sound levels at the ears. A benefit of multiple loudspeakers in the configuration of the array described here is that by delivering test sounds (usually bands of noise) from all seven loudspeakers a quasi-diffuse noise region is created around the listener's location. Head movements within this region of normal head movements can be shown to result in much less level variation than when the sound is presented from a single loudspeaker. More stable sound levels allow reliable threshold measurements to 16 kHz.

Another application is the demonstration of directional hearing aids. When introducing a patient to the features of new hearing aids, audiologists frequently demonstrate the benefit of directional microphones. This is typically done by delivering target speech from a straight-ahead loudspeaker and interfering noise from a loudspeaker directly behind. This can be a very misleading demonstration because the directional microphones are often most effective in reducing sounds for rearward sources, but much less effective from other directions. Switching from omnidirectional to directional microphones will produce a much larger noise reduction than would typically be achieved in real-world conditions. The array described here can give a fairer demonstration. By positioning the listener so that the array is behind, with an eighth loudspeaker delivering speech straight-ahead of the listener, the degree of noise reduction from directional microphones that is demonstrated to the listener will be more representative of what can be expected in normal use conditions.

Another application is the simulation of reverberant rooms. The spatial extent of the seven-loudspeaker array provides a sufficient range of interaural differences for the creation of realistic simulations of reverberant spaces, for a listener at the center listening position. Creating such a multi-source reverberant room simulation (MRRS) first involves specifying the acoustic characteristics of the room to be simulated, along with the sources and listener location and the acoustic absorption coefficients of each of the six surfaces. In one possible approach, an image-method simulation is constructed in which a simulated directional microphone is placed at the listener's location. This directional microphone is pointed in the direction of each of the sound sources in the array [−60, −30, −15, 0, 15, 30, and 60 deg], along with their supplementary angles [−120, −150, −165, −180, 165, 150, and 120 deg]. Simulated responses from each source direction and its supplementary direction (e.g., 30 and 150 deg) are summed to form the response from the source direction (e.g., 30 deg). Inclusion of the supplementary angle augments the direct response by taking advantage of the approximate front/back symmetry of acoustic responses around the head. In this way, every source/listener configuration in the specified reverberant room will have a seven-channel impulse response that will approximate the responses from those directions in the simulation. In the present application, however, the possible source directions are those of the seven loudspeakers in the array, and the only listener location is the center of the array. A simulation is created by filtering source signals with the seven-channel impulse responses and delivering them from the array. The simulation conveys the impression of listening in a reverberant space. In audiometric testing, this method can be used for adding controlled degrees of reverberation to any type of sound field test for enhanced realism.

In other applications the system can be used to test spatial aspects of hearing when listeners use any of a number of head-worn devices, including hearing aids, cochlear implants, hearing protectors, and helmets.

### 5 Alternatives

In general, as the user moves relative to the system, larger than natural effects on sound levels may occur. In some examples, the system tracks the user's head as it moves and compensates for those effects.

In some examples, a camera or other suitable sensor is used to track the position of the user's head relative to the testing position. For example, other vision, ultrasonic, or radio-frequency localization devices may be used to track the subject's head position.

In some examples, an eighth loudspeaker is located behind the user's head for testing localization ability for sources located behind the user. In yet other examples, a three-dimensional arrangement of loudspeakers can be used to assess vertical as well as horizontal spatial hearing characteristics.

In some examples, the system includes a calibration microphone and performs a calibration test (on demand or periodically or on an ongoing basis during testing) to adapt the system to the acoustic environment (e.g., by pinging reference levels from the loudspeakers and comparing the recorded levels to previously determined calibration levels).

In some examples, the cabinet includes magnets that are used to fully or partially suspend the cabinet on the walls of a sound booth, which are often perforated-steel walls.

In some examples, the sensor data used by the feedback system described above can be used to control the stimuli emitted by the system as a test or experiment progresses. For example, sound levels at the various loudspeakers can be adjusted using the sensor data that indicates a position of the user's head (e.g., levels are reduced as the user's head comes closer to the loudspeaker and vice-versa).

### 6 Implementations

The approaches described above can be implemented, for example, using a programmable computing system executing suitable software instructions or it can be implemented in suitable hardware such as a field-programmable gate array (FPGA) or in some hybrid form. For example, in a programmed approach the software may include procedures in one or more computer programs that execute on one or more programmed or programmable computing system (which may be of various architectures such as distributed, client/server, or grid) each including at least one processor, at least one data storage system (including volatile and/or non-volatile memory and/or storage elements), at least one user interface (for receiving input using at least one input device or port, and for providing output using at least one output device or port). The software may include one or more modules of a larger program, for example, that provides services related to the design, configuration, and execution of dataflow graphs. The modules of the program (e.g., elements of a dataflow graph) can be implemented as data structures or other organized data conforming to a data model stored in a data repository.

The software may be stored in non-transitory form, such as being embodied in a volatile or non-volatile storage medium, or any other non-transitory medium, using a physical property of the medium (e.g., surface pits and lands, magnetic domains, or electrical charge) for a period of time (e.g., the time between refresh periods of a dynamic memory device such as a dynamic RAM). In preparation for loading the instructions, the software may be provided on a tangible, non-transitory medium, such as a CD-ROM or other computer-readable medium (e.g., readable by a general or special purpose computing system or device), or may be delivered (e.g., encoded in a propagated signal) over a communication medium of a network to a tangible, non-transitory medium of a computing system where it is executed. Some or all of the processing may be performed on a special purpose computer, or using special-purpose hardware, such as coprocessors or field-programmable gate arrays (FPGAs) or dedicated, application-specific integrated circuits (ASICs). The processing may be implemented in a distributed manner in which different parts of the computation specified by the software are performed by different computing elements. Each such computer program is preferably stored on or downloaded to a computer-readable storage medium (e.g., solid state memory or media, or magnetic or optical media) of a storage device accessible by a general or special purpose programmable computer, for configuring and operating the computer when the storage device medium is read by the computer to perform the processing described herein. The inventive system may also be considered to be implemented as a tangible, non-transitory medium, configured with a computer program, where the medium so configured causes a computer to operate in a specific and predefined manner to perform one or more of the processing steps described herein.

In general, the controller of the spatial hearing measurement system includes computing devices (e.g., general purpose computers and/or single board microcontrollers) that interface with and control various other components of the system. For example, an audio output signal is formed by a computing device and output through a sound card (including a digital to analog converter) to an amplifier. The amplifier amplifies the output signal and provides the amplified signal to one or more of the loudspeakers, which transduce the signal. Similarly, signals sensed by the sensors and the microphones are digitized using an analog to digital converter. The digitized sensor signals are provided to the computing device for processing. The computing devices include outputs for displaying data to a user and inputs for receiving user input.

A number of embodiments of the invention have been described. Nevertheless, it is to be understood that the foregoing description is intended to illustrate and not to limit the scope of the invention, which is defined by the scope of the following claims. Accordingly, other embodiments are also within the scope of the following claims. For example, various modifications may be made without departing from the scope of the invention. Additionally, some of the steps described above may be order independent, and thus can be performed in an order different from that described.

What is claimed is:

1. A measurement system comprising:
a plurality of audio transducers configured in a first arrangement for acoustic testing of a subject in a testing position;
one or more sensors for collecting measurement data characterizing a position of a subject's head relative to the plurality of audio transducers;
a controller for processing the measurement data to determine feedback data characterizing a difference between the position of the subject's head and the testing position; and
a feedback indicator for presenting the feedback data to the subject.

2. The system of claim 1 wherein the plurality of audio transducers is arrayed on a horizontal plane intersecting with the testing position.

3. The system of claim 2 wherein the audio transducers are arranged symmetrically over a range of between ±15 degrees and ±30 degrees relative to a 0 degree angle relative to the testing position.

4. The system of claim 3 wherein the plurality of audio transducers are spaced apart with:
a first audio transducer oriented with a 0 degree angle relative to the testing position;
second and third audio transducers oriented with a ±15 degree angle relative to the testing position;
fourth and fifth audio transducers oriented with a ±30 degree angle relative to the testing position; and
sixth and seventh audio transducers oriented with a ±60 degree angle relative to the testing position.

5. The system of claim 1 wherein a first sensor of the one or more sensors is configured to sense the left-hand side of the subject's head, a second sensor of the one or more sensors is configured to sense the right-hand side of the subject's head, and a third sensor is configured to sense a front side of the subject's head.

6. The system of claim 1 wherein the one or more sensors includes one or more optical distance measurement devices.

7. The system of claim 1 wherein the feedback indicator is configurable to provide controllable patterns of illumination.

8. The system of claim 7, wherein the feedback indicator includes an LED array.

9. The system of claim 7 wherein the feedback data presented to the subject indicates actions that the subject can take to reduce the difference between their head position and the testing position.

10. The system of claim 1 wherein the controller is configured to conduct a test when the subject's head is in the testing position, the test including causing emission of acoustic energy from at least some of the plurality of transducers.

11. The system of claim 10 wherein causing emission of the acoustic energy includes supplying one or more signals to the at least some transducers.

12. The system of claim 11 wherein the controller is configured to modify the one or more signals to simulate reverberation in the emitted acoustic energy.

13. The system of claim 1, wherein the measurement system is sized and shaped such that the plurality of audio transducers fit in a sound booth.

14. The system of claim 1, wherein the plurality of audio transducers comprises a plurality of loudspeakers.

15. A method for testing spatial hearing, comprising:
sensing, using one or more sensors, a position of a subject's head relative to a plurality of audio transducers configured in a first arrangement for acoustic testing of the subject in a testing position
providing feedback to the subject based on the sensing of the position;
providing acoustic stimuli from loudspeakers of the measurement system; and
receiving responses to the acoustic stimuli from the subject.
